SEQUENCE LISTING (1 sequence)

SEQ ID NO: 1 - DNA, 11 bp, Artificial Sequence
  Synthetic DNA
acgtcatgata

(19) United States
(12) Patent Application Publication
(10) Pub. No.: US 2021/0210164 A1
(43) Pub. Date: Jul. 8, 2021

(54) SYSTEMS AND METHODS FOR MAPPING SEQUENCE READS

(71) Applicant: LIFE TECHNOLOGIES CORPORATION, Carlsbad, CA (US)

(72) Inventors: Zheng ZHANG, Arcadia, CA (US); Fiona HYLAND, San Mateo, CA (US); Sowmi UTIRAMERUR, Pleasanton, CA (US)

(21) Appl. No.: 17/136,800

(22) Filed: Dec. 29, 2020

Related U.S. Application Data
(63) Continuation of application No. 15/679,261, filed on Aug. 17, 2017, now abandoned.
Application No. 15/679,261 is a division of application No. 13/363,717, filed on Feb. 1, 2012, now abandoned.
(60) Provisional application No. 61/483,442, filed on May 6, 2011.
(60) Provisional application No. 61/446,427, filed on Feb. 24, 2011.
(60) Provisional application No. 61/438,545, filed on Feb. 1, 2011.

(51) Int. Cl. G16B 30/00

(52) U.S. Cl. CPC G16B 30/00

(57) ABSTRACT

Systems, methods, and computer program products for aligning a fragment sequence to a target sequencing. The alignment is allowed at most one gap, such as an insertion or a deletion. In some embodiments, both a gapped alignment and an ungapped alignment can be produced. A selection can be made between the gapped alignment and the ungapped alignment based on a quality value for each alignment.

### RELATED APPLICATIONS

This application is related to U.S. Provisional Application No. 61/438,545 filed Feb. 1, 2011 which is incorporated herein by reference in its entirety and U.S. Provisional Application No. 61/446,427 filed Feb. 24, 2011, which is incorporated herein by reference in its entirety and U.S. Provisional Application No. 61/483,442 filed May 6, 2011, which is incorporated herein by reference in its entirety.

### FIELD

The present disclosure relates to biomolecule sequencing, and in particular to systems and methods for mapping sequence reads.

### INTRODUCTION

Nucleic acid sequence information can be an important data set for medical and academic research endeavors. Sequence information can facilitate medical studies of active disease and genetic disease predispositions, and can assist in rational design of drugs (e.g., targeting specific diseases, avoiding unwanted side effects, improving potency, and the like). Sequence information can also be a basis for genomic and evolutionary studies and many genetic engineering applications. Reliable sequence information can be critical for other uses of sequence data, such as paternity tests, criminal investigations and forensic studies.

Sequencing technologies and systems, such as, for example, those provided by Applied Biosystems/Life Technologies (SOLiD Sequencing System), Solexa (Illumina), and 454 Life Sciences (Roche) can provide high throughput DNA/RNA sequencing capabilities to the masses. Applications which may benefit from these sequencing technologies include, but are certainly not limited to, targeted resequencing, miRNA analysis, DNA methylation analysis, whole-transcriptome analysis, and cancer genomics research.

Sequencing platforms can vary from one another in their mode of operation (e.g., sequencing by synthesis, sequencing by ligation, pyrosequencing, etc.) and the type/form of raw sequencing data that they generate. Generally, however, sequencing systems incorporating NGS technologies can produce a large number of short reads. As a result, these sequencing systems must be able to map a large number of reads against a genome in a relatively short amount of time. For a human size genome, for example, a sequencing system must map billions of reads.

### SUMMARY

In various embodiments, a processor can map fragment sequences to a target sequence. Additionally, the processor can identify short insertions or deletions within the fragment sequences. These and other features are provided herein.

### BRIEF DESCRIPTION OF THE DRAWINGS

The skilled artisan will understand that the drawings, described below, are for illustration purposes only. The drawings are not intended to limit the scope of the present teachings in any way.

FIG. 1 is a diagram illustrating an exemplary deletion.

FIG. 2 is a diagram illustrating an exemplary insertion.

FIG. 3 is a flow diagram illustrating an exemplary embodiment of a method of a method of aligning a fragment sequence to a reference sequence

FIG. 4 is a flow diagram illustrating another exemplary embodiment of a method of aligning a fragment sequence to a reference sequence.

FIG. 5 is a block diagram that illustrates a computer system, in accordance with various embodiments.

FIG. 6 is a block diagram that illustrates a system for determining a nucleic acid sequence, in accordance with various embodiments.

FIG. 7 is a plot illustrating the number of insertions or deletions identified at various lengths.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts.

### DESCRIPTION OF VARIOUS EMBODIMENTS

The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings. In this application, the use of the singular includes the plural unless specifically stated otherwise. Also, the use of “comprise”, “comprises”, “comprising”, “contain”, “contains”, “containing”, “include”, “includes”, and “including” are not intended to be limiting. It is to be understood that both the foregoing general description and the following detailed description are exemplary and explanatory only and are not restrictive of the present teachings.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and techniques described herein are those well known and commonly used in the art.

As used herein, “a” or “an” may also refer to “at least one” or “one or more”. Further, unless expressly stated to the contrary, “or” refers to an inclusive-or and not to an exclusive-or. For example, a condition A or B is satisfied by any one of the following: A is true (or present) and B is false (or not present), A is false (or not present) and B is true (or present), and both A and B are true (or present).

The phrase “next generation sequencing” refers to sequencing technologies having increased throughput as compared to traditional Sanger- and capillary electrophoresis-based approaches, for example with the ability to generate hundreds of thousands of relatively small sequence reads at a time. Some examples of next generation sequencing techniques include, but are not limited to, sequencing by synthesis, sequencing by ligation, and sequencing by hybridization. More specifically, the SOLiD Sequencing System of Life Technologies Corp. provides massively parallel sequencing with enhanced accuracy. The SOLiD System and associated workflows, protocols, chemistries, etc. are described in more detail in PCT Publication No. WO 2006/084132, entitled “Reagents, Methods, and Libraries for Bead-Based Sequencing,” international filing date Feb. 1, 2006, U.S. patent application Ser. No. 12/873,190, entitled “Low-Volume Sequencing System and Method of Use,” filed on Aug. 31, 2010, and U.S. patent application Ser. No. 12/873,132, entitled “Fast-Indexing Filter Wheel and Method of Use,” filed on Aug. 31, 2010, the entirety of each of these applications being incorporated herein by reference thereto. Additionally, the Personal Genome Machine (PGM) of Life Technologies Corp. provides massively parallel sequencing with enhanced accuracy. The PGM System and associated workflows, protocols, chemistries, etc. are described in more detail in U.S. Patent Application Publication No. 2009/0127589 and No. 2009/0026082, the entirety of each of these applications being incorporated herein by reference.

The phrase “sequencing run” refers to any step or portion of a sequencing experiment performed to determine some information relating to at least one biomolecule (e.g., nucleic acid molecule).

The phrase “ligation cycle” refers to a step in a sequence-by-ligation process where a probe sequence is ligated to a primer or another probe sequence.

The phrase “color call” refers to an observed dye color that results from the detection of a probe sequence after a ligation cycle of a sequencing run. Similarly, other “calls” refer to the distinguishable feature observed.

The phase “base space” refers to a representation of the sequence of nucleotides. The phase “flow space” refers to a representation of the incorporation event or non-incorporation event for a particular nucleotide flow. For example, flow space can be a series of zeros and ones representing a nucleotide incorporation event (a one, “1”) or a non-incorporation event (a zero, “0”) for that particular nucleotide flow. It should be understood that zeros and ones are convenient representations of a non-incorporation event and a nucleotide incorporation event; however, any other symbol or designation could be used alternatively to represent and/or identify these events and non-events.

DNA (deoxyribonucleic acid) is a chain of nucleotides consisting of 4 types of nucleotides; A (adenine), T (thymine), C (cytosine), and G (guanine), and that RNA (ribonucleic acid) is comprised of 4 types of nucleotides; A, U (uracil), G, and C. Certain pairs of nucleotides specifically bind to one another in a complementary fashion (called complementary base pairing). That is, adenine (A) pairs with thymine (T) (in the case of RNA, however, adenine (A) pairs with uracil (U)), and cytosine (C) pairs with guanine (G). When a first nucleic acid strand binds to a second nucleic acid strand made up of nucleotides that are complementary to those in the first strand, the two strands bind to form a double strand. As used herein, “nucleic acid sequencing data,” “nucleic acid sequencing information,” “nucleic acid sequence,” “genomic sequence,” “genetic sequence,” or “fragment sequence,” or “nucleic acid sequencing read” denotes any information or data that is indicative of the order of the nucleotide bases (e.g., adenine, guanine, cytosine, and thymine/uracil) in a molecule (e.g., whole genome, whole transcriptome, exome, oligonucleotide, polynucleotide, fragment, etc.) of DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic signature-based systems, etc.

A “polynucleotide”, “nucleic acid”, or “oligonucleotide” refers to a linear polymer of nucleosides (including deoxyribonucleosides, ribonucleosides, or analogs thereof) joined by internucleosidic linkages. Typically, a polynucleotide comprises at least three nucleosides. Usually oligonucleotides range in size from a few monomeric units, e.g. 3-4, to several hundreds of monomeric units. Whenever a polynucleotide such as an oligonucleotide is represented by a sequence of letters, such as “ATGCCTG,” it will be understood that the nucleotides are in 5′->3′ order from left to right and that “A” denotes deoxyadenosine, “C” denotes deoxycytidine, “G” denotes deoxyguanosine, and “T” denotes thymidine, unless otherwise noted. The letters A, C, G, and T may be used to refer to the bases themselves, to nucleosides, or to nucleotides comprising the bases, as is standard in the art.

The phrase “fragment library” refers to a collection of nucleic acid fragments, wherein one or more fragments are used as a sequencing template. A fragment library can be generated, for example, by cutting or shearing a larger nucleic acid into smaller fragments. Fragment libraries can be generated from naturally occurring nucleic acids, such as bacterial nucleic acids. Libraries comprising similarly sized synthetic nucleic acid sequences can also be generated to create a synthetic fragment library.

The phrase “paired-end library” refers to a collection of nucleic acid fragments, wherein one or more fragments are used as a sequencing template to obtain sequence information from both ends of the fragment. A paired-end library can be generated, for example, by cutting or shearing a larger nucleic acid into smaller fragments. Paired-end libraries can be generated from naturally occurring nucleic acids, such as bacterial nucleic acids. Libraries comprising similarly sized synthetic nucleic acid sequences can also be generated to create a synthetic fragment library.

The phrase “mate-pair library” refers to a collection of nucleic acid sequences comprising two fragments having a relationship, such as by being separated by a known number of nucleotides. Mate pair fragments can be generated by cutting or shearing, or they can be generated by circularizing fragments of nucleic acids with an internal adapter construct and then removing the middle portion of the nucleic acid fragment to create a linear strand of nucleic acid comprising the internal adapter with the sequences from the ends of the nucleic acid fragment attached to either end of the internal adapter. Like fragment libraries, mate-pair libraries can be generated from naturally occurring nucleic acid sequences. Synthetic mate-pair libraries can also be generated by attaching synthetic nucleic acid sequences to either end of an internal adapter sequence.

The term “template” and variations thereof refer to a nucleic acid sequence that is a target of nucleic acid sequencing. A template sequence can be attached to a solid support, such as a bead, a microparticle, a flow cell, or other surface or object. A template sequence can comprise a synthetic nucleic acid sequence. A template sequence also can include an unknown nucleic acid sequence from a sample of interest and/or a known nucleic acid sequence.

The phrase “template density” refers to the number of template sequences attached to each individual solid support.

In various embodiments, a sequence alignment method can align a fragment sequence to a reference sequence or another fragment sequence. The fragment sequence can be obtained from a fragment library, a paired-end library, a mate-pair library, or another type of library that may be reflected or represented by nucleic acid sequence information including for example, RNA, DNA, and protein based sequence information. Generally, the length of the fragment sequence can be substantially less than the length of the reference sequence. The fragment sequence and the reference sequence can each include a sequence of symbols. The alignment of the fragment sequence and the reference sequence can include a limited number of mismatches between the symbols of the fragment sequence and the symbols of the reference sequence. Generally, the fragment sequence can be aligned to a portion of the reference sequence in order to minimize the number of mismatches between the fragment sequence and the reference sequence.

In particular embodiments, the symbols of the fragment sequence and the reference sequence can represent the composition of biomolecules. For example, the symbols can correspond to identity of nucleotides in a nucleic acid, such as RNA or DNA, or the identity of amino acids in a protein. In some embodiments, the symbols can have a direct correlation to these subcomponents of the biomolecules. For example, each symbol can represent a single base of a polynucleotide. In other embodiments, each symbol can represent two or more adjacent subcomponent of the biomolecules, such as two adjacent bases of a polynucleotide. Additionally, the symbols can represent overlapping sets of adjacent subcomponents or distinct sets of adjacent subcomponents. For example, when each symbol represents two adjacent bases of a polynucleotide, two adjacent symbols representing overlapping sets can correspond to three bases of polynucleotide sequence, whereas two adjacent symbols representing distinct sets can represent a sequence of four bases. Further, the symbols can correspond directly to the subcomponents, such as nucleotides, or they can correspond to a color call or other indirect measure of the subcomponents. For example, the symbols can correspond to an incorporation or non-incorporation for a particular nucleotide flow.

In various embodiments, a sequence alignment method can produce a gapped semi-local alignment, in which the fragment sequence is fully aligned and the reference sequence may not be fully aligned. The gapped semi-local alignment can include a gap in the alignment of the fragment sequence to the reference sequence. The gap can include an insertion into the fragment sequence or a deletion from the fragment sequence. In particular embodiments, the gapped semi-local alignment can have at most one gap in the alignment. Further, the gap can conform to certain requirements, such as a maximum length of a deletion or a maximum length of an insertion.

In various embodiments, the sequence alignment method can match an anchor portion of the fragment sequence to a portion of the reference sequence. The anchor portion can include a contiguous portion of the fragment sequence. The anchor portion of the reference sequence can be an approximate match to a portion of the reference sequence, including, for example, a small number of mismatches between the anchor portion and the reference sequence. In various embodiments, the anchor portion can have a length that is not greater than half the length of the fragment sequence. In particular embodiments, the anchor portion can have a length of at least one quarter of the length of the fragment sequence. Further, the sequence alignment method can extend the alignment from the anchor portion to substantially the entire length of the fragment sequence.

In various embodiments, a sequence alignment method can select from an ungapped local alignment and a gapped alignment. The ungapped local alignment can align the fragment sequence to the reference sequence without a gap in the alignment. A mapping quality value can be determined for each of the gapped alignment and the ungapped local alignment, and the mapping quality values can be compared to select the better alignment of the fragment sequence to the reference sequence.

In various embodiments, a computer program product can include instructions to select a contiguous portion of a fragment sequence; instructions to map the contiguous portion of the fragment sequence to a reference sequence using an approximate string matching method that produces at least one match of the contiguous portion to the reference sequence; instructions to map a gap containing portion of the fragment sequence to the reference sequence using a gapped alignment method that produces an alignment of the gap containing portion extending from the contiguous portion to complete a map of the fragment sequence, the gap containing portion including at most one insertion or deletion.

In various embodiments, a system for nucleic acid sequence analysis can include a data analysis unit. The data analysis unit can be configured to obtain a fragment sequence from a sequencing instrument, obtain a reference sequence, select a contiguous portion of the fragment sequence, and map the contiguous portion of the fragment sequence to the reference sequence using an approximate string mapping method that produces at least one match of the contiguous portion to the reference sequence. The data analysis unit can be further configured to map a remaining portion of the read to reference sequence using an ungapped local alignment method that produces an ungapped local alignment extending from the at least one match, and determine if the ungapped local alignment extends substantially an entire length of the fragment sequence. When the ungapped local alignment does not extend substantially the entire length of the sequence fragment, the data analysis unit can map a gap containing portion of the fragment sequence to the reference sequence using a gapped alignment method that produces a gapped alignment extending from the at least one match. The gap containing portion including at most one insertion or deletion. The data analysis unit can determine a first quality value for the ungapped local alignment and a second quality value for the gapped alignment, and select from the ungapped local alignment and the gapped alignment based on the first quality value and the second quality value.

FIG. 1 illustrates an exemplary deletion in a fragment sequence. When reference sequence 102 is aligned with fragment sequence 104, a portion 106 of the reference sequence 102 can be seen to be missing or deleted from fragment sequence 104. Plot 108 provides another illustration of the deletion. In plot 108, the vertical axis 110 represents the position of a nucleotide in the fragment sequence and the horizontal axis 112 represents the position of a nucleotide in the reference sequence. For a given alignment, when the position of each symbol of the fragment sequence is plotted versus the position of the corresponding symbol of the reference sequence, the resulting line 114 is generated. Within the deleted region 116, line 114 is horizontal, indicating an advancement along the reference sequence without a corresponding advancement along the fragment sequence.

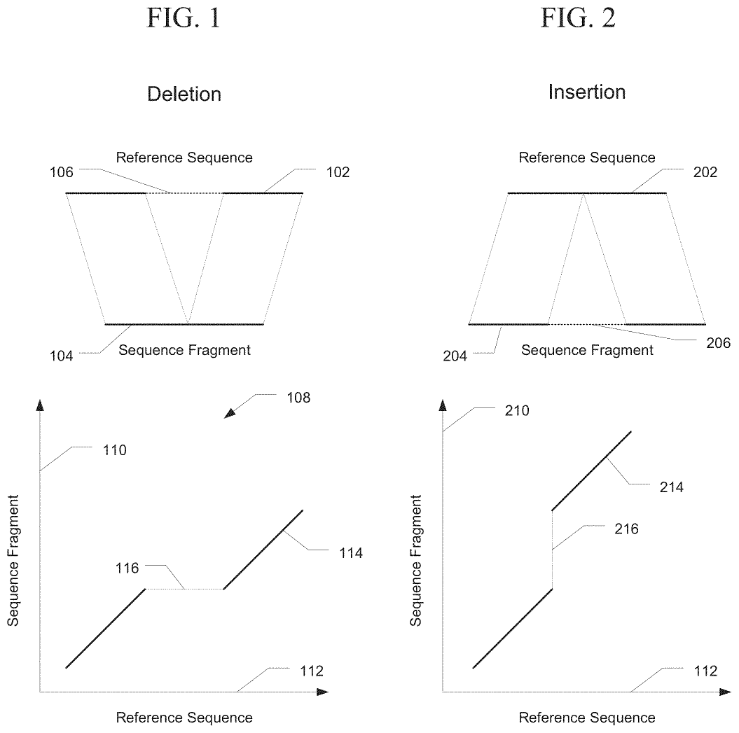

FIG. 2 illustrates an exemplary insertion in a fragment sequence. When reference sequence 202 is aligned with fragment sequence 204, a portion 206 can be seen to be added or inserted into the fragment sequence 204. Plot 208 provides another illustration of the insertion. In plot 208, the vertical axis 210 represents the position of a nucleotide in the fragment sequence and the horizontal axis 212 represents the position of a nucleotide in the reference sequence. For a given alignment, the position of each symbol of the fragment sequence is plotted versus the position of the corresponding symbol of the reference sequence, the resulting line 214 is generated. Within the inserted region 216, line 214 is vertical, indicating advancement along the fragment sequence without a corresponding advancement along the reference sequence.

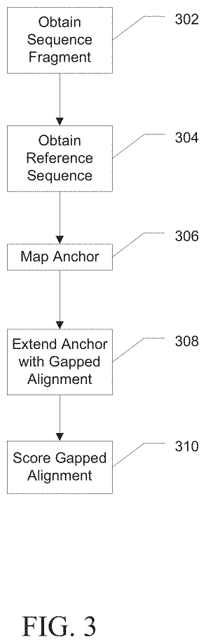

FIG. 3 illustrates an exemplary method for aligning a fragment sequence to a reference sequence. At 302, a fragment sequence can be obtained. In various embodiments, the fragment sequence can have a length of greater than about 40, such as at least about 50 symbols. Additionally, the fragment sequence can have a length not greater than about 5000 symbols, such as not greater than about 2000 symbols, such as not greater than about 1000 symbols, such as not greater than about 500 symbols, such as not greater than about 250 symbols, such as not greater than about 150 symbols, even not greater than about 75 symbols. At 304, a reference sequence can be obtained. In various embodiments, the symbols can represent base calls, color calls, flow space information, or the like.

At 306, an anchor portion of the fragment sequence can be matched against the reference sequence using an approximate string mapping technique. The anchor portion can be a contiguous portion of the fragment sequence that can be mapped to the reference sequence. For example, a portion of the reference sequence can be identified that substantially matches the sequence of the anchor portion while allowing for a limited number of mismatches. In various embodiments, the length of the anchor portion can be less than half the length of the fragment sequence.

In particular embodiments, an anchor portion from a first half of the fragment sequence can be mapped to the reference sequence, or an anchor portion from a second half of the fragment sequence can be mapped to the reference sequence. Significantly, in order to match the reference sequence, the portion of the fragment sequence selected as the anchor portion does not span a gap in the alignment. Further, as the gap will generally be located in either the first half or the second half, a matching anchor portion can be chosen to be in the other half from the gap. In an example, an attempt can be made to match portions from the first half of the fragment sequence to the reference sequence in order to find an anchor portion. If unsuccessful, an attempt may be made to match portions from the second half of the sequence to the reference sequence in order to find an anchor portion.

At 308, after an anchor portion has been identified that maps to the reference sequence, the anchor portion can be extended along the length of the fragment sequence using a gapped alignment method. In various embodiments, the gapped alignment method can allow for at most one gap in the alignment of the fragment sequence to the reference sequence. The gap can be an insertion into the fragment sequence or a deletion from the fragment sequence. Additionally, the length of the gap can be set within a specified threshold or limited to a maximum length. In various embodiments, a deletion can be set within a specified threshold or have a maximum deletion length and an insertion can be set within a specified threshold or have a maximum insertion length, and the maximum deletion length and the maximum insertion length may not necessarily be the same. For example, the maximum insertion and the maximum deletion lengths can be in a range of about 2 to about 20. In particular embodiments, the maximum insertion length can be in a range of 2 to about 7, such as a maximum length of about 4. In particular embodiments, the maximum deletion length can be in a range of about 7 to about 15, such as about 11.

At each position, a decision can be made to extend the aligned portion of the sequence, initiate a gap at the location, or extend the gap when the gap length will not exceed the maximum gap length or theshold. A score of a gapped alignment can be calculated using a scoring function. For example, the scoring function can be defined by score=M+mx+G, where M is the number of matches in the extended alignment, x is the number of mismatches in the extended alignment, and m is a score for each mismatch, and G is a score for a gap satisfying the size restriction. In various embodiments, the extension step can select a gapped alignment having the best score from possible alignments having, for example, at most one gap satisfying the gap size restriction.

In particular embodiments, parameters that can affect the alignment can include the location of the anchor on the read, the length of the anchor and the maximum number of allowed mismatches, the maximum size of the insertion or deletion, a minimum length of an aligned portion after the insertion or deletion, and a maximum length of an unaligned portion of the read.

For example, given a fragment sequence “ACGTCGACA” and a reference sequence “ACGTCATGATA”, an anchor portion of the fragment sequence “ACGT” (shown in bold) can be selected and aligned with the reference sequence.

| | |
|---|---|
| Fragment | ACGT |
| Reference | ACGTCATGATA |

After the anchor portion “ACGT” is aligned to the reference, the anchor portion can be extended. The resulting gapped alignment can identify an “AT” deletion between position 5 and 6 (indicated as “-”) in the fragment sequence and a mismatch (indicated in lowercase) at position 8 of the fragment sequence.

| | |
|---|---|
| Fragment | ACGTC--GAcA |
| Reference | ACGTCATGAtA |

Alternatively, without allowing for a gap, there would be a significant number of mismatches spanning the gap and beyond. As such, the resulting alignment may only include the bases up to the gap.

| | |
|---|---|
| Fragment | ACGTCgaca |
| Reference | ACGTCatgaTA |

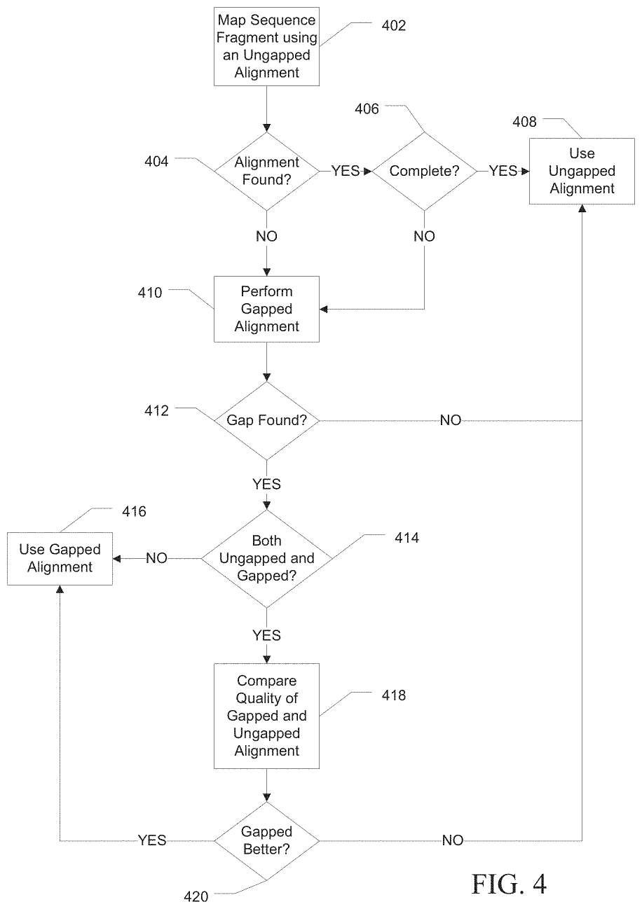

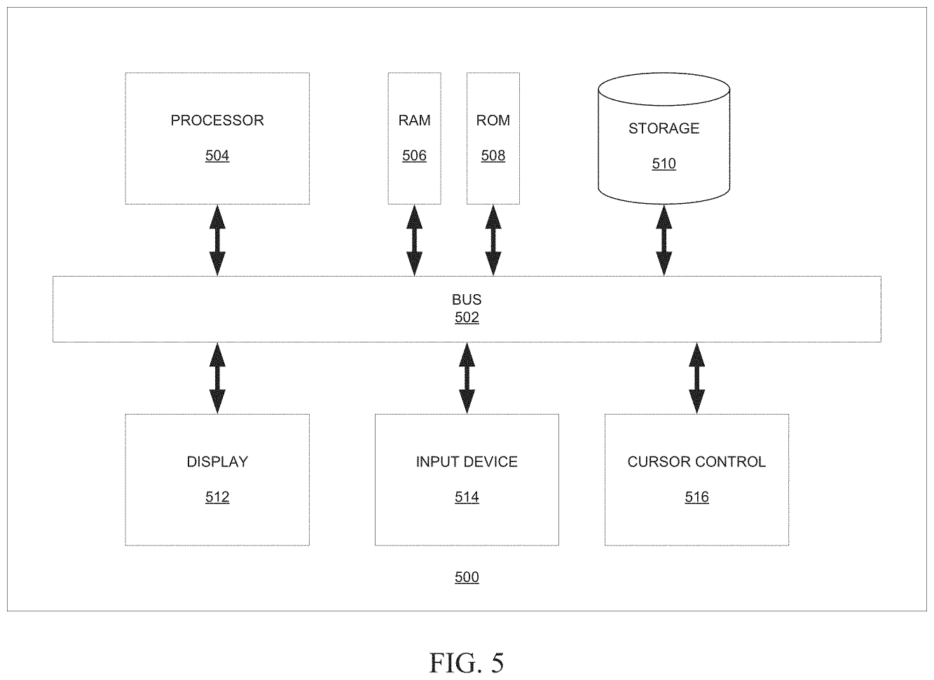

FIG. 4 illustrates another exemplary method for aligning a fragment sequence to a reference sequence. At 402, a fragment sequence can be mapped to the reference sequence using an ungapped alignment method. An ungapped alignment method can be used to identify the longest portion of the fragment sequence that corresponds to a contiguous portion of the reference sequence without allowing for a gap in the alignment. Using the ungapped local alignment method, a proportion of the fragment sequence can be matched against the reference sequence using an approximate string mapping technique. For example, a portion of the reference sequence can be identified that substantially matches the sequence of the anchor portion while allowing for a limited number of mismatches. In particular embodiments, the anchor portion can have an approximated length not greater than one half the length of fragment sequence. Additionally, the anchor portion can have an approximated length at least one quarter the length of the fragment sequence.

In particular embodiments, once the anchor portion is mapped to the reference sequence, the alignment can be extended along the length of the fragment sequence. A score of an extended alignment can be calculated using a scoring function. For example, the scoring function can be defined by score=M+mx, where M is the number of matches in the extended alignment, x is the number of mismatches in the extended alignment, and m is score for each mismatch. According to the scoring function, each match can be given a score of one and each mismatch can be given a mismatch score, m, such as a negative penalty for a mismatch.

In various embodiments, the extension step can select an extended alignment having the best score from all possible extended alignments. Significantly, the extended alignment with the best score may not extend the full length of the fragment sequence. For example, when an end portion of the fragment sequence does not match the corresponding portion of the reference sequence, the best ungapped alignment may exclude the end portion of the fragment sequence since including the additional mismatches can reduce the overall score of the alignment.

At 404, it can be determined if an alignment is found. When the alignment is found, it can be determined if the alignment is substantially complete, as shown at 406. The alignment can be determined to be substantially complete when the alignment extends substantially the entire length of the fragment sequence, such as at least 75% of the length, such as at least 80% of the length, such as at least 85% of the length, even at least 95% of the length. When the alignment is determined to be substantially complete, the ungapped alignment can be reported as the alignment of the fragment sequence to the reference sequence, as shown at 408.

Alternatively, when and ungapped alignment is not found, or when the ungapped alignment is not substantially complete, a gapped alignment method can be performed, as shown at 410. As previously described, the gapped alignment method can permit, for example, at most one gap having a length not greater than a maximum length. In particular embodiments, the gap can be a deletion having a length not greater than a maximum deletion length or an insertion having a length not greater than a maximum insertion length. A score of a gapped alignment can be calculated using a gapped scoring function. For example, the gapped scoring function can be defined by score=M+mx+G, where M is the number of matches in the extended alignment, x is the number of mismatches in the extended alignment, and m is a score for each mismatch, and G is a score for a gap satisfying the size restriction. For example, the gapped alignment method can select from a gapped alignment having the best score from all possible gapped alignments having at most one gapped with a length not greater than the maximum gap length.

At 412, it can be determined if a gapped alignment is found. When a gap alignment is not found, the ungapped alignment can be reported, as shown at 408. Alternatively, when a gapped alignment is found, it can be determined if both a gapped alignment and an ungapped alignment have been identified, as shown at 414. When only a gapped alignment is found, the gapped alignment can be reported as the alignment of the fragment sequence to the reference sequence, as shown at 416.

Alternatively, when both gapped and ungapped alignments are found, the quality of the gapped alignment can be compared to the quality of the ungapped alignment, as shown at 418. For example, a quality value can be calculated for each of the gapped alignment and the ungapped alignment. The quality values for the gapped and ungapped alignments can be compared to determine which alignment is better, such as which alignment is more complete, has fewer mismatches, is less likely to result from an incorrect alignment, or combinations thereof.

In various embodiments, a quality value can be calculated for each of the gapped and ungapped alignments. The quality value can depend on the size of the insertion or deletion, the length of the alignments on either side of the gap, and a total number of mismatches in the aligned portions. Further, the quality value can be calculated by determining the probability that the identified alignment is a correct alignment. For example, the quality value can be determined by calculating a Bayesian posterior probability score. P(r|A)_{InDel} and P_{PartialAlignment} can be calculated where A is the predicted gapped alignment and the null hypothesis is the longest partial alignment either side of the gap. The calculation can model the likelihood that the predicted gapped alignment is the actual alignment of the fragment sequence to the reference sequence. In an example, a posterior probability for the alignment can be calculated by

$${P ( A \vert r )}_{InDel} = \frac{{P ( r \vert A )}_{InDel}}{{P ( r \vert A )}_{InDel} + {P}_{PartialAlignment}} ,$$

where A is the event that fragment sequence r aligns with the identified region of the reference sequence, and the partial alignment for the alternative hypothesis is the longer of the alignments either side of the insertion or deletion.

At 420, it can be determined if the gapped alignment has a higher quality than the ungapped alignment, such as when the gapped alignment has a higher probability of being a correct alignment. When the gapped alignment is better than the ungapped alignment, the gapped alignment can be reported, as shown at 416. Alternatively, with gapped alignment is not better than the ungapped alignment, the ungapped alignment can be reported, as shown at 408.

FIG. 5 is a block diagram that illustrates a computer system 500, upon which embodiments of the present teachings can be implemented. Computer system 500 can include a bus 502 or other communication mechanism for communicating information, and a processor 504 coupled with bus 502 for processing information. Computer system 500 can also include a memory 506, which can be a random access memory (RAM) or other dynamic storage device, coupled to bus 502. Memory 506 can store data, such as sequence information, and instructions to be executed by processor 504. Memory 506 can also be used for storing temporary variables or other intermediate information during execution of instructions to be executed by processor 504. Computer system 500 can further include a read-only memory (ROM) 508 or other static storage device coupled to bus 502 for storing static information and instructions for processor 504. A storage device 510, such as a magnetic disk, an optical disk, a flash memory, or the like, can be provided and coupled to bus 502 for storing information and instructions.

Computer system 500 can be coupled by bus 502 to display 512, such as a cathode ray tube (CRT) or liquid crystal display (LCD), for displaying information to a computer user. An input device 514, such as a keyboard including alphanumeric and other keys, can be coupled to bus 502 for communicating information and commands to processor 504. Cursor control 516, such as a mouse, a trackball, a trackpad, or the like, can communicate direction information and command selections to processor 504, such as for controlling cursor movement on display 512. The input device can have at least two degrees of freedom in at least two axes that allows the device to specify positions in a plane. Other embodiments can include at least three degrees of freedom in at least three axes to allow the device to specify positions in a space. In additional embodiments, functions of input device 514 and cursor 516 can be provided by a single input devices such as a touch sensitive surface or touch screen.

Computer system 500 can perform the present teachings. Consistent with certain implementations of the present teachings, results are provided by computer system 500 in response processor 504 executing one or more sequences of one or more instructions contained in memory 506. Such instructions may be read into memory 506 from another computer-readable medium, such as storage device 510. Execution of the sequences of instructions contained in memory 506 can cause processor 504 to perform the processes described herein. Alternatively, hard-wired circuitry may be used in place of or in combination with software instructions to implement the present teachings. Thus, implementations of the present teachings are not limited to any specific combination of hardware circuitry and software.

The term “computer-readable medium” as used herein refers to any media that participates in providing instructions to processor 504 for execution. Such a medium may take many forms, including but not limited to, nonvolatile memory, volatile memory, and transmission media. Nonvolatile memory includes, for example, optical or magnetic disks, such as storage device 510. Volatile memory includes dynamic memory, such as memory 506. Transmission media includes coaxial cables, copper wire, and fiber optics, including the wires that comprise bus 502. Non-transitory computer readable medium can include nonvolatile media and volatile media.

Common forms of non-transitory computer readable media include, for example, floppy disk, flexible disk, hard disk, magnetic tape, or any other magnetic medium, a CD-ROM, any other optical medium, punch cards, paper tape, any other physical medium with patterns of holes, a RAM, a PROM, an EPROM, a FLASH-EPROM, and other memory chips or cartridge or any other tangible medium from which the computer can read.

Various forms of computer readable media may be involved in carrying one or more sequences of one or more instructions to processor 504 for execution. For example the instructions may initially be stored on the magnetic disk of a remote computer. The remote computer can load the instructions into its dynamic memory and send instructions over a network to computer system 500. A network interface coupled to bus 502 can receive the instructions and place the instructions on bus 502. Bus 502 can carry the instructions to memory 506, from which processor 504 can retrieve and execute the instructions. Instructions received by memory 506 may optionally be stored on storage device 510 either before or after execution by processor 504.

Nucleic acid sequence data can be generated using various techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic signature-based systems, etc.

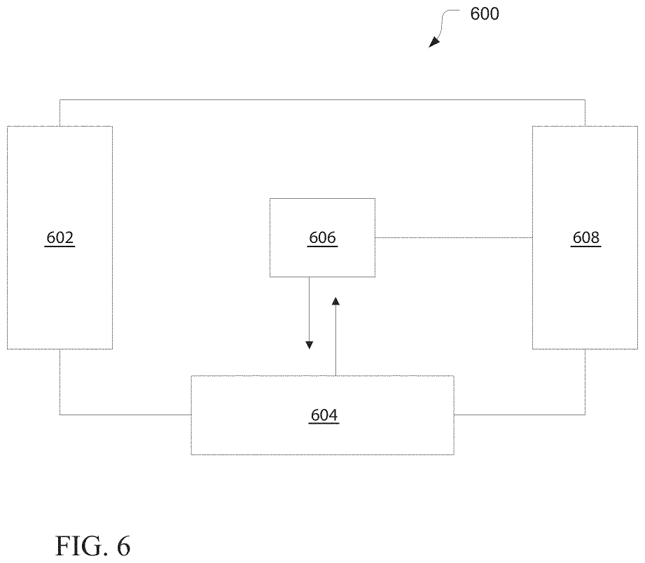

Various embodiments of nucleic acid sequencing platforms, such as a nucleic acid sequencer, can include components as displayed in the block diagram of FIG. 6. According to various embodiments, sequencing instrument 600 can include a fluidic delivery and control unit 602, a sample processing unit 604, a signal detection unit 606, and a data acquisition, analysis and control unit 608. Various embodiments of instrumentation, reagents, libraries and methods used for next generation sequencing are described in U.S. Patent Application Publication No. 6009/0127589 and No. 6009/0026082 are incorporated herein by reference. Various embodiments of instrument 600 can provide for automated sequencing that can be used to gather sequence information from a plurality of sequences in parallel, such as substantially simultaneously.

In various embodiments, the fluidics delivery and control unit 602 can include reagent delivery system. The reagent delivery system can include a reagent reservoir for the storage of various reagents. The reagents can include RNA-based primers, forward/reverse DNA primers, oligonucleotide mixtures for ligation sequencing, nucleotide mixtures for sequencing-by-synthesis, optional ECC oligonucleotide mixtures, buffers, wash reagents, blocking reagent, stripping reagents, and the like. Additionally, the reagent delivery system can include a pipetting system or a continuous flow system which connects the sample processing unit with the reagent reservoir.

In various embodiments, the sample processing unit 604 can include a sample chamber, such as flow cell, a substrate, a micro-array, a multi-well tray, or the like. The sample processing unit 604 can include multiple lanes, multiple channels, multiple wells, or other means of processing multiple sample sets substantially simultaneously. Additionally, the sample processing unit can include multiple sample chambers to enable processing of multiple runs simultaneously. In particular embodiments, the system can perform signal detection on one sample chamber while substantially simultaneously processing another sample chamber. Additionally, the sample processing unit can include an automation system for moving or manipulating the sample chamber.

In various embodiments, the signal detection unit 606 can include an imaging or detection sensor. For example, the imaging or detection sensor can include a CCD, a CMOS, an ion or chemical sensor, such as an ion sensitive layer overlying a CMOS or FET, a current or voltage detector, or the like. The signal detection unit 606 can include an excitation system to cause a probe, such as a fluorescent dye, to emit a signal. The excitation system can include an illumination source, such as arc lamp, a laser, a light emitting diode (LED), or the like. In particular embodiments, the signal detection unit 606 can include optics for the transmission of light from an illumination source to the sample or from the sample to the imaging or detection sensor. Alternatively, the signal detection unit 606 may provide for electronic or non-photon based methods for detection and consequently not include an illumination source. In various embodiments, electronic-based signal detection may occur when a detectable signal or species is produced during a sequencing reaction. For example, a signal can be produced by the interaction of a released byproduct or moiety, such as a released ion, such as a hydrogen ion, interacting with an ion or chemical sensitive layer. In other embodiments a detectable signal may arise as a result of an enzymatic cascade such as used in pyrosequencing (see, for example, U.S. Patent Application Publication No. 6009/0325145, the entirety of which being incorporated herein by reference) where pyrophosphate is generated through base incorporation by a polymerase which further reacts with ATP sulfurylase to generate ATP in the presence of adenosine 5′ phosphosulfate wherein the ATP generated may be consumed in a luciferase mediated reaction to generate a chemiluminescent signal. In another example, changes in an electrical current can be detected as a nucleic acid passes through a nanopore without the need for an illumination source.

In various embodiments, a data acquisition analysis and control unit 608 can monitor various system parameters. The system parameters can include temperature of various portions of instrument 600, such as sample processing unit or reagent reservoirs, volumes of various reagents, the status of various system subcomponents, such as a manipulator, a stepper motor, a pump, or the like, or any combination thereof.

It will be appreciated by one skilled in the art that various embodiments of instrument 600 can be used to practice variety of sequencing methods including ligation-based methods, sequencing by synthesis, single molecule methods, nanopore sequencing, and other sequencing techniques.

In various embodiments, the sequencing instrument 600 can determine the sequence of a nucleic acid, such as a polynucleotide or an oligonucleotide. The nucleic acid can include DNA or RNA, and can be single stranded, such as ssDNA and RNA, or double stranded, such as dsDNA or a RNA/cDNA pair. In various embodiments, the nucleic acid can include or be derived from a fragment library, a mate pair library, a ChIP fragment, or the like. In particular embodiments, the sequencing instrument 600 can obtain the sequence information from a single nucleic acid molecule or from a group of substantially identical nucleic acid molecules.

In various embodiments, sequencing instrument 600 can output nucleic acid sequencing read data in a variety of different output data file types/formats, including, but not limited to: *.fasta, *.csfasta, *seq.txt, *qseq.txt, *.fastq, *.sff, *prb.txt, *.sms, *srs and/or *.qv.

In accordance with various embodiments, instructions configured to be executed by processor to perform a method are stored on a computer readable medium. The computer readable medium can be a device that stores digital information. For example, a computer readable medium can include a compact disc read-only memory as is known in the art for storing software. The computer readable medium is accessed via processor suitable for executing instructions configured to be executed.

In a first aspect, a method of nucleic acid sequence analysis can include receiving nucleic acid sequence information comprising a fragment sequence and nucleic acid sequence information comprising at least one reference sequence. The method can further include selecting a contiguous portion of the fragment sequence, mapping the contiguous portion of the fragment sequence to the reference sequence using an approximate string matching method that produces at least one match of the contiguous portion to the reference sequence, and mapping a gap containing portion of the fragment sequence to the reference sequence using a gapped alignment method that produces an alignment of the gap containing portion extending from the contiguous portion to complete a map of the fragment sequence. The gap containing portion can include at most one insertion or deletion.

In an exemplary embodiment, the method can further include extending the contiguous portion of the fragment sequence using an ungapped local alignment method.

In an exemplary embodiment, the method can further include selecting a contiguous portion of the fragment sequence and maps the contiguous portion of the reference sequence iteratively. In a particular embodiment, the method can further include selecting a contiguous portion at a different location but with the same length on the read at each iteration until the at least one match is produced. In a particular embodiment, the method can further include selecting a contiguous portion at a same location but with a different length on the read at each iteration until a number of matches of the contiguous portion to the reference sequence is less than a certain threshold.

In an exemplary embodiment, the alignment can extend from the at least one match in either direction.

In an exemplary embodiment, the gap containing portion can include one insertion having a length less than a maximum insertion length.

In an exemplary embodiment, the gap containing portion can include one deletion having a length less than a maximum deletion length.

In an exemplary embodiment, the gapped alignment method uses a scoring function and selects an alignment with the best score. In a particular embodiment, the scoring function can be a sum of a product of a number of matches and a match score, a product of a number of mismatches and a mismatch score, and a gap score.

In embodiments of the first aspect, the method can further include mapping a remaining portion of the read to reference sequence using an ungapped local alignment method that produces an ungapped local alignment extending from the at least one match. In particular embodiments, the method can further include determining if the ungapped local alignment extends substantially an entire length of the fragment sequence. In particular embodiments, the method can further include determining a first quality value for the ungapped local alignment and a second quality value for the gapped alignment, and selecting from the ungapped local alignment and the gapped alignment based on the first quality value and the second quality value.

In a second aspect, a system for nucleic acid sequence analysis can include a data analysis unit. The data analysis unit can be configured to obtain a fragment sequence from a sequencing instrument and obtain a reference sequence. The data analysis unit can be further configured to select a contiguous portion of the fragment sequence, and map the contiguous portion of the fragment sequence to the reference sequence using an approximate string mapping method that produces at least one match of the contiguous portion to the reference sequence. The data analysis unit can be further configured to map a remaining portion of the read to reference sequence using an ungapped local alignment method that produces an ungapped local alignment extending from the at least one match, and determine if the ungapped local alignment extends substantially an entire length of the fragment sequence. The data analysis unit can be configured to map a gap containing portion of the fragment sequence to the reference sequence using a gapped alignment method that produces a gapped alignment extending from the at least one match when the ungapped local alignment does not extend substantially the entire length of the fragment sequence. The gap containing portion can include at most one insertion or deletion. The data analysis unit can be configured to determine a first quality value for the ungapped local alignment and a second quality value for the gapped alignment, and select from the ungapped local alignment and the gapped alignment based on the first quality value and the second quality value when both a ungapped local alignment and a gapped alignment are identified.

In an exemplary embodiment, the data analysis unit can be further configured to select a contiguous portion of the fragment sequence and maps the contiguous portion of the reference sequence iteratively. In a particular embodiment, the data analysis unit can be further configured to select a contiguous portion at a different location but with the same length on the read at each iteration until the at least one match is produced. In a particular embodiment, the data analysis unit can be further configured to select a contiguous portion at a same location but with a different length on the read at each iteration until a number of matches of the contiguous portion to the reference sequence is less than a certain threshold.

In an exemplary embodiment, the alignment can extend from the at least one match in either direction. In a particular embodiment, the gap containing portion can include one insertion having a length less than a maximum insertion length. In a particular embodiment, the gap containing portion can include one deletion having a length less than a maximum deletion length.

In an exemplary embodiment, the gapped alignment method uses a gapped alignment scoring function and selects an alignment with the best score. In a particular embodiment, the gapped alignment scoring function is a sum of a product of a number of matches and a match score, a product of a number of mismatches and a mismatch score, and a gap score.

In an exemplary embodiment, the ungapped local alignment method uses an ungapped alignment scoring function and selects an alignment with the best score. In a particular embodiment, the ungapped alignment scoring function is a sum of a number of matches and a product of a number of mismatches and a mismatch score.

In a third aspect, a computer program product can include a non-transitory computer-readable storage medium whose contents include a program with instructions to be executed on a processor. The instructions can include instructions to select a contiguous portion of a fragment sequence; instructions to map the contiguous portion of the fragment sequence to a reference sequence using an approximate string matching method that produces at least one match of the contiguous portion to the reference sequence; and instructions to map a gap containing portion of the fragment sequence to the reference sequence using a gapped alignment method that produces an alignment of the gap containing portion extending from the contiguous portion to complete a map of the fragment sequence. The gap containing portion including at most one insertion or deletion.

In an exemplary embodiment, the instructions can further include instructions to extend the contiguous portion of the fragment sequence using an ungapped local alignment method.

In an exemplary embodiment, the instructions can further include instructions to select a contiguous portion of the fragment sequence and maps the contiguous portion of the reference sequence iteratively. In a particular embodiment, the instructions can further include instructions to select a contiguous portion at a different location but with the same length on the read at each iteration until the at least one match is produced. In a particular embodiment, the instructions can further include instructions to select a contiguous portion at a same location but with a different length on the read at each iteration until a number of matches of the contiguous portion to the reference sequence is less than a certain threshold.

In an exemplary embodiment, the alignment can extend from the at least one match in either direction.

In an exemplary embodiment, the gap containing portion can include one insertion having a length less than a maximum insertion length.

In an exemplary embodiment, the gap containing portion can include one deletion having a length less than a maximum deletion length.

In an exemplary embodiment, the gapped alignment method can use a scoring function and selects an alignment with the best score. In a particular embodiment, the scoring function can include a sum of a product of a number of matches and a match score, a product of a number of mismatches and a mismatch score, and a gap score.

While the principles of the present teachings have been described in connection with specific embodiments of control systems and sequencing platforms, it should be understood clearly that these descriptions are made only by way of example and are not intended to limit the scope of the present teachings or claims. What has been disclosed herein has been provided for the purposes of illustration and description. It is not intended to be exhaustive or to limit what is disclosed to the precise forms described. Many modifications and variations will be apparent to the practitioner skilled in the art. What is disclosed was chosen and described in order to best explain the principles and practical application of the disclosed embodiments of the art described, thereby enabling others skilled in the art to understand the various embodiments and various modifications that are suited to the particular use contemplated. It is intended that the scope of what is disclosed be defined by the following claims and their equivalents.

Further, in describing various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments.

The embodiments described herein, can be practiced with other computer system configurations including hand-held devices, microprocessor systems, microprocessor-based or programmable consumer electronics, minicomputers, mainframe computers and the like. The embodiments can also be practiced in distributing computing environments where tasks are performed by remote processing devices that are linked through a network.

It should also be understood that the embodiments described herein can employ various computer-implemented operations involving data stored in computer systems. These operations are those requiring physical manipulation of physical quantities. Usually, though not necessarily, these quantities take the form of electrical or magnetic signals capable of being stored, transferred, combined, compared, and otherwise manipulated. Further, the manipulations performed are often referred to in terms, such as producing, identifying, determining, or comparing.

Any of the operations that form part of the embodiments described herein are useful machine operations. The embodiments, described herein, also relate to a device or an apparatus for performing these operations. The systems and methods described herein can be specially constructed for the required purposes or it may be a general purpose computer selectively activated or configured by a computer program stored in the computer. In particular, various general purpose machines may be used with computer programs written in accordance with the teachings herein, or it may be more convenient to construct a more specialized apparatus to perform the required operations.

Certain embodiments can also be embodied as computer readable code on a computer readable medium. The computer readable medium is any data storage device that can store data, which can thereafter be read by a computer system. Examples of the computer readable medium include hard drives, network attached storage (NAS), read-only memory, random-access memory, CD-ROMs, CD-Rs, CD-RWs, magnetic tapes, and other optical and non-optical data storage devices. The computer readable medium can also be distributed over a network coupled computer systems so that the computer readable code is stored and executed in a distributed fashion.

### Examples

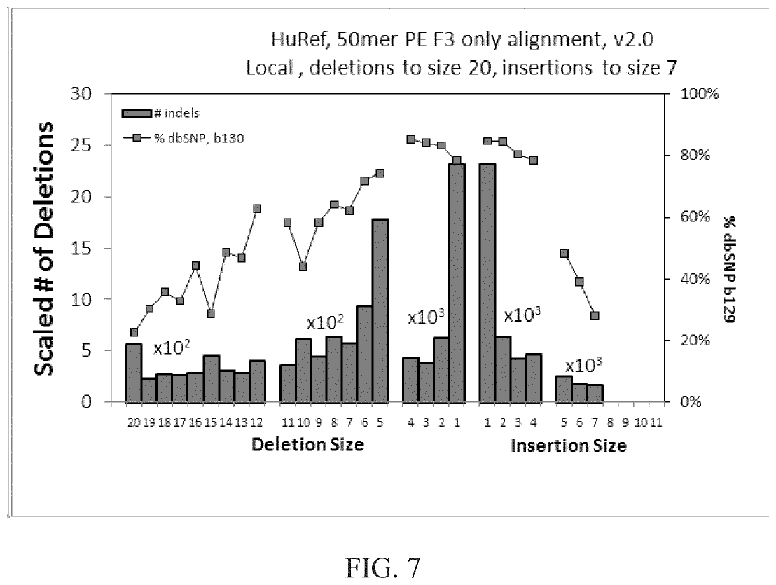

FIG. 7 shows the results of a comparison of a paired-end library data set derived from HuRef and the HG18 reference genome.

1.-34. (canceled)

35. A method of analyzing a nucleic acid fragment sequence for an alignment with a reference nucleic acid sequence, wherein the fragment sequence is produced by a nucleic acid sequencing instrument in response to detecting a plurality of signals representative of at least a portion of a sequence of at least one nucleic acid fragment, the method comprising:
receiving the fragment sequence and at least one reference sequence at a processor, wherein the fragment sequence comprises a sequence of symbols representing nucleotides in the nucleic acid fragment and the reference sequence comprises a sequence of symbols representing nucleotides in a reference nucleic acid;
selecting a contiguous portion of the fragment sequence;
mapping the contiguous portion of the fragment sequence to the reference sequence using an approximate string matching method to produce an at least partial match of the contiguous portion to the reference sequence;
mapping a remaining portion extending from the contiguous portion of the fragment sequence to the reference sequence using an ungapped local alignment method to produce an ungapped alignment extending from the contiguous portion, the ungapped local alignment method comprising calculating an ungapped alignment score based on a number of ungapped alignment matches and a number of ungapped alignment mismatches for a given alignment length, and identifying an optimal alignment for the ungapped alignment based on the ungapped alignment score at each alignment length;
mapping the remaining portion extending from the contiguous portion of the fragment sequence to the reference sequence using a gapped alignment method to produce a gapped alignment of the remaining portion extending from the contiguous portion, wherein the gapped alignment method includes calculating a gapped alignment score for a given gapped alignment by calculating a sum of a number of gapped alignment matches, a product of a number of gapped alignment mismatches and a gapped alignment mismatch score, and a gap score, and identifying the gapped alignment corresponding to a best gapped alignment score;
determining a first quality value for the ungapped alignment and a second quality value for the gapped alignment;
comparing the first quality value and the second quality value to determine a higher quality value; and
selecting one of the ungapped alignment and the gapped alignment corresponding to the higher quality value to identify a best alignment of the fragment sequence and the reference sequence for a report.

36. The method of claim 35, wherein the selecting a contiguous portion of the fragment sequence and the mapping the contiguous portion of the fragment sequence to the reference sequence are performed in one or more iterations.

37. The method of claim 36, wherein the selecting a contiguous portion includes selecting contiguous portions each at a different location and having a same length on the fragment sequence at each iteration.

38. The method of claim 36, wherein the selecting a contiguous portion includes selecting contiguous portions each at a same location and having a different length on the fragment sequence at each iteration.

39. The method of claim 35, wherein the gapped alignment extends from the at least partial match in either direction.

40. The method of claim 35, wherein the remaining portion extending from the contiguous portion of the fragment sequence includes a gap containing portion, the gap containing portion including an insertion or deletion.

41. The method of claim 40, wherein the gap containing portion includes one insertion having a length less than a maximum insertion length.

42. The method of claim 40, wherein the gap containing portion includes one deletion having a length less than a maximum deletion length.

43. The method of claim 35, further comprising determining if the ungapped alignment extends substantially an entire length of the fragment sequence.

44. A system for analyzing a nucleic acid fragment sequence for an alignment with a reference nucleic acid sequence, wherein the fragment sequence is produced by a nucleic acid sequencing instrument in response to detecting a plurality of signals representative of at least a portion of a sequence of at least one nucleic acid fragment,
the system comprising:
a processor configured to:
receive the fragment sequence from the nucleic acid sequencing instrument, wherein the fragment sequence comprises a sequence of symbols representing nucleotides in the nucleic acid fragment;
obtain at least one reference sequence, wherein the reference sequence comprises a sequence of symbols representing nucleotides in a reference nucleic acid;
select a contiguous portion of the fragment sequence;
map the contiguous portion of the fragment sequence to the reference sequence using an approximated string mapping method to produce an at least partial match of the contiguous portion to the reference sequence;
map a remaining portion extending from the contiguous portion of the fragment sequence to the reference sequence using an ungapped local alignment method to produce an ungapped alignment extending from the contiguous portion, the ungapped local alignment method comprising calculating an ungapped alignment score based on a number of ungapped alignment matches and a number of ungapped alignment mismatches for a given alignment length, and identifying an optimal alignment for the ungapped alignment based on the ungapped alignment score at each alignment length;
map the remaining portion extending from the contiguous portion of the fragment sequence to the reference sequence using a gapped alignment method to produce a gapped alignment of the remaining portion extending from the contiguous portion, the gapped alignment method including calculating a gapped alignment score by calculating a sum of a number of gapped alignment matches, a product of a number of gapped alignment mismatches and a gapped alignment mismatch score, and a gap score, and identifying the gapped alignment corresponding to a best gapped alignment score;
determine a first quality value for the ungapped alignment and a second quality value for the gapped alignment;
compare the first quality value and the second quality value to determine a higher quality value; and
select one of the ungapped alignment and the gapped alignment corresponding to the higher quality value to identify a best alignment of the fragment sequence and the reference sequence for a report.

45. The system of claim 44, wherein the processor is further configured to select a contiguous portion of the fragment sequence and map the contiguous portion to the reference sequence in one or more iterations.

46. The system of claim 45, wherein the processor is further configured to select contiguous portions each at a different location and having a same length on the fragment sequence at each iteration.

47. The system of claim 45, wherein the processor is further configured to select contiguous portions each at a same location and having a different length on the fragment sequence at each iteration.

48. The system of claim 44, wherein the gapped alignment extends from the at least partial match in either direction.

49. The system of claim 44, wherein the remaining portion extending from the contiguous portion of the fragment sequence includes a gap containing portion, the gap containing portion including an insertion or deletion.

50. The system of claim 49, wherein the gap containing portion includes one insertion having a length less than a maximum insertion length.

51. The system of claim 49, wherein the gap containing portion includes one deletion having a length less than a maximum deletion length.

52. The system of claim 44, wherein the processor is configured to calculate a sum of the number of ungapped alignment matches and a product of the number of ungapped alignment mismatches and an ungapped alignment mismatch score to determine the ungapped alignment score.

53. A computer program product, comprising a non-transitory computer-readable storage medium whose contents include a program with instructions for execution by a processor, the instructions comprising:
instructions to obtain a fragment sequence, the fragment sequence produced by a nucleic acid sequencing instrument in response to detecting a plurality of signals representative of at least a portion of a sequence of at least one nucleic acid fragment, wherein the fragment sequence comprises a sequence of symbols representing nucleotides in the nucleic acid fragment;
instructions to obtain at least one reference sequence, wherein the reference sequence comprises a sequence of symbols representing nucleotides in a reference nucleic acid;
instructions to select a contiguous portion of a fragment sequence;
instructions to map the contiguous portion of the fragment sequence to a reference sequence using an approximated string matching method to produce an at least partial match of the contiguous portion to the reference sequence;
instructions to map a remaining portion extending from the contiguous portion of the fragment sequence to the reference sequence using an ungapped local alignment method to produce an ungapped alignment extending from the contiguous portion, the ungapped local alignment method comprising calculating an ungapped alignment score based on a number of ungapped alignment matches and a number of ungapped alignment mismatches for a given alignment length and identifying an optimal alignment for the ungapped alignment based on the ungapped alignment score at each alignment length;
instructions to map the remaining portion extending from the contiguous portion of the fragment sequence to the reference sequence using a gapped alignment method to produce a gapped alignment of the remaining portion extending from the contiguous portion, the gapped alignment method including calculating a gapped alignment score by calculating of a number of gapped alignment matches, a product of a number of gapped alignment mismatches and a gapped alignment mismatch score, and a gap score, and identifying the gapped alignment corresponding to a best gapped alignment score;
instructions to determine a first quality value for the ungapped alignment and a second quality value for the gapped alignment;
instructions to compare the first quality value and the second quality value to determine a higher quality value; and
instructions to select one of the ungapped alignment and the gapped alignment corresponding to the higher quality value to identify a best alignment of the fragment sequence and the reference sequence for a report.

54. The computer program product of claim 53, further comprising instructions to select a contiguous portion of the fragment sequence and map to the contiguous portion of the reference sequence in one or more interations.
